(19) United States
(12) Patent Application Publication
(10) Pub. No.: US 2021/0030580 A1
(43) Pub. Date: Feb. 4, 2021

(54) BIO-ENGINEERED WEARABLE DEVICE FOR PROMOTING HEALTHY CERVICAL SPINAL POSTURE AND MUSCLE MEMORY WHILE LIMITING CONCERNS OVER MUSCULOSKELETAL DEVELOPMENT IN BOTH CHILDREN AND ADULTS WHEN USING HANDHELD ELECTRONIC MEDIA DEVICES

(71) Applicant: Richard M. Targett, Moraga, CA (US)

(72) Inventor: Richard M. Targett, Moraga, CA (US)

(21) Appl. No.: 16/530,308

(22) Filed: Aug. 2, 2019

(51) Int. Cl. A61F 5/37

(52) U.S. Cl. CPC A61F 5/37

(57) ABSTRACT

A wearable bio-engineered device for preventing cervical spine and other musculoskeletal injuries in humans. The device includes a support frame having a pair of spaced apart support arm members that gently engage about the sides of the human neck of the wearer; and a chin support member supported by said support frame and disposed beneath the chin and above the chest of the wearer. The support frame and chin support member are configured and sized so that when the head of the human wearer flexes downward towards the chest of the wearer, while viewing the handheld mobile device, the chin support member is positioned directly between the chin and chest of the wearer and physically constrains and limits the neck from flexing and head from tilting beyond a predetermined flexion limit set by the physical length dimensions of the spaced apart support arm members and the physical height dimension of the chin support member.

### BACKGROUND OF THE INVENTION

### Field of Invention

The present invention is directed to a wearable device for children and adults alike to reduce the risk of developing musculoskeletal and cervical spine issues caused by prolonged use of electronic handheld devices, cellphones, laptops, portable computers and television monitors.

### Brief Description of the State of the Art

Mobile devices and portable computing devices are necessary for communication, entertainment and general productivity. Also, tablets, smart cellphones and laptops are more readily available to children with recent studies identifying large increases in daily screen time usage worldwide amongst all age groups.

Studies have reported that the visual display terminal of a tablet PC, which has a smaller screen than that of a regular desktop computer, requires the user to bend his/her neck more. The angle at which someone looks at portable small screen devices is lower than that occurring with a big screen. When someone constantly bends their head to look at a small screen, various problems, such as a forward-head posture or a slouched, turtle-like posture, can arise. Therefore, the use of mobile handheld computers and smart phones can be associated with greater head and neck flexion and shorter viewing distances than desktop computers. This poses all sorts of risks to developing spinal, musculoskeletal and cervical issues.

It is well known that healthy cervical vertebrae have a forward convex curve. However, during the use of portable electronic devices, the user inclines forward. This extends the muscle behind the neck too much, placing a substantial load and stress on the cervical vertebrae. This abnormal posture, together with the changes in the mechanism of contraction, places pressure on the facet joint and disks, causing headaches and neck pain. The use of visual display terminals is closely related to increased fatigue of the neck and arm muscles, and musculoskeletal problems.

FIGS. 1A through 1E illustrates various positions when the human head is tilted from 0 to 60 degrees of neck/head flexion angle, while viewing a handheld mobile device such as a smartphone, and generating strain on the spinal vertebrae along the neck of the human skeletal system. During these different viewing positions, varying increases in weight and pressure are imposed on the cervical spine as the neck, particularly the chin position in relation to a neutral position, is in a forward flexion position. The depiction of both the gaze angle and the forward flexion of the neck is represented by the fixed position of the handheld device. By eliminating changes in the position of the handheld device isolates the forward flexion position to vary accordingly.

It is further noted the eye gaze angle (field of vision) will vary by individual. Most notably, those wearing eyeglasses will exhibit more forward flexion position in order to keep the glass lenses centered on the screen. Thus, adults and children who wear glasses are further at risk of increased forward neck flexion due to the restriction on their ability to shift their eye gaze downward as their field of vision will then be outside the field of vision area of the lenses.

FIG. 2A shows the human skeleton where the cervical spine (cervical vertebrae c1 to c7) are in the neutral position, where the head is centered over the shoulders aligning the cervical and thoracic spine. In this neutral position, the chin is parallel to the ground. This position does not exert any additional pressure on the musculoskeletal structure of the body. The head constitutes approximately 6% of the total body weight, which is linked to the cervical spine and all other joints through the kinematic chain by various muscles. Normal posture is defined as when the line of gravity (log) passes through the external auditory meatus, the bodies of the cervical spine, and the acromion and anterior to the thoracic spine.

FIG. 2B illustrates that cervical vertebrae c1 to c7 are restricted and carry the increased weight and pressure along with the musculoskeletal frame when the human tilts his/her head towards a 15 degree flexion angle. The closer the chin is moved toward the chest (from neutral 90 degree parallel in the neutral position), results in excessive forward flexion, and has deleterious consequences for the musculoskeletal system when repeated over a prolonged time period.

As shown in FIG. 2B, failure of the head to align with the vertical axis of the body, can lead to further malalignments in the body, namely, rounded shoulders and increased thoracic, compensate kyphosis to for the altered location of the log, leading to further impairments. Combination of all these postural deviations is often known as “slouched posture or “slumped posture.” The thoracic spine is less mobile and, thus, more stable in comparison to the cervical region. This stability is attributed to the presence of ribs, lesser intervertebral disk, and the plane of facet joints.

FIGS. 3A and 3B illustrate a human viewing a laptop computer system on a desktop at different viewing angles, illustrating how the human head is tilted, during viewing and creating stress and strain along the vertebrae of the human neck and spinal column. From this illustration, it can be shown that the forward neck flexion angle of the viewer will is more severe when viewing a handheld device positioned near the laptop keyboard, than when viewing the display screen of the laptop on a flat desk like surface.

In view of the above discussion, there is mounting evidence that the forward head flexion observed during handheld electronic device use has increased the demands on the body's musculoskeletal structure to compensate for the extended periods of time spent daily on these devices. This is most notably apparent in the cervical section of the spine and surrounding musculature anatomy.

This is of particular concern with young children (ages 5-15) who are in the active physical musculoskeletal growth phase, where the potential impact of increased forward neck flexion for long periods has not been determined. In addition, there is no clear indication of the impact these handheld devices may have when held close to the mid torso region during active and resting use. Handheld mobile devices connected via cellular or WIFI connectivity are sending and receiving signals even during a restful state. In addition, there is no conclusionary evidence of the impact on vision due to the prolonged gaze angle necessary to view the handheld mobile device screens.

With, normal head posture, stress on the neck is minimized because the head's weight is naturally balanced on the cervical spine. Forward head posture occurs when the neck slants forward, placing the head further in front of the shoulders rather than directly aligned. This head position can lead to multiple problems caused by prolonged increased stress on the cervical spine in the forward head flexion position. As the head is held forward, the cervical spine and musculoskeletal structure must support the increasing amounts of weight. As a rule of thumb, for every inch that the head is held forward in poor posture, an additional 10 pounds of weight is felt on the cervical spine. So if the average head weighs between 10 and 12 pounds, just 1 or 2 inches of forward head posture can double or triple the load on the cervical spine and surrounding musculoskeletal structure, which can lead to significant adverse results.

Concerns Over Hyperflexion and Hyperextension. The lower cervical spine goes into hyperflexion with the vertebrae tilting too far forward. The upper cervical spine, however, does the opposite and goes into hyperextension as the brain automatically keeps the head up so the eyes can look straight ahead. This alteration of the cervical spine's curve lengthens the spinal canal distance from the base of the skull to the base of the neck, causing the spinal cord and nearby nerve roots to become somewhat stretched.

Concerns Over Muscle overload. Some muscles in the neck and upper back must continually overwork to counterbalance the pull of gravity on the forward head. As a result, muscles become more susceptible to painful strains and spasms. Hunched upper back. Forward head posture is often accompanied by forward shoulders and a rounded upper back, which can lead to more pain in the neck, upper back, and/or shoulders. The longer that poor posture is continued—such as being hunched over a computer or slouching on the couch—the more likely that neck pain, stiffness, and other symptoms may develop. Over time, forward head posture can put increasing amounts of stress on the neck and other areas of the body. Some long-term effects of forward head posture can include:

Concerns Over Muscle imbalances. Some muscles in the neck, upper back, shoulders, and chest can become shortened and tight, whereas others can become elongated and weak. Elevated risk for spinal degeneration. Extra stress on the cervical spine's discs, facet joints, and vertebrae may increase the risk for or worsen degenerative spine issues, such as cervical degenerative disc disease and cervical osteoarthritis. Reduced mobility. With increased stiffness in the muscles and/or joints, the neck's range of motion is decreased.

To date, many ergonomic studies and devices have addressed the effects of long hours working on desktop computers, though not applicable from the effects of the use of handheld mobile devices and the human skeletal system. The resulting effects on eye strain, lower back, neck pain and carpal tunnel complications have all been evaluated and devices and procedures have been put into practice to help address and minimize this well-researched problem. Awareness of the effects from long duration desktop usage is keen, while the growing use of handheld electronic devices that are not used or set up like a desktop have received less attention.

Maintaining the neutral posture position has been a primary goal of most ergonomic devices developed for desktop computer usage. Notably, however, handheld electronic devices or devices held in the lap generally do not benefit from these ergonomic devices and methods developed for desktop computing.

Handheld devices force the eye gaze and neck angle to be accelerated in the downward position creating the problem that the present invention intends to minimize. Chronic head-forward-flexion can lead to several issues including disc herniations, degenerative disc disease, early onset arthritis, bone spurs, and radiating numbness and pain traveling down the arm.

Currently, there are few known long-term studies that have evaluated the impact of handheld electronic device use on a growing musculoskeletal anatomy in a developing child where regular and extended periods in the forward held flexion position caused by the use of such handheld mobile device. One research study conducted in Australia (Straker, Harris, 2000) noted that 60% of children in their study reported discomfort while using their laptop. With increased usage of cell phones, tablets and laptop computers, this leaves the neck more vulnerable to overuse injuries and along with the smaller screens on the devices, makes it more likely the neck area is to susceptible to injury. When the head is in forward flexion for extended periods of time can put 3 to 5 times more demand on the joints, discs, and musculature of the neck compared to a normalized neutral posture where the head sits directly over the shoulders. Chronic head-forward-posture can lead to several issues including disc herniations, degenerative disc disease, early onset arthritis, bone spurs, and radiating numbness and pain traveling down the arm.

Studies have shown that neck flexion angle of 15 degrees or more can put 2 to 3 times more stress on the neck compared to neutral posture. Effects on children from chronic neck flexion can be severe. One condition is Scheuermann's Disease. Scheuermann's disease is a condition where children develop thoracic spine anterior wedging of the vertebrae from increased force being put on the anterior portion of the vertebrae while the growth plates in the bones are still developing. This ultimately causes permanent deformity where the shoulders are rounded forward with the head coming forward. Habit and awareness play a part with forward head flexion too.

A 2017 study done by Ailneni, et. al at the University of Northern Illinois proved that creating a device which increased awareness of posture ultimately helped subjects to work with better neck posture. It does not come easy for most individuals to be aware of their posture, which is what inspired the (name of devise). By increasing the population's awareness when the forward head flexion and neck posture go beyond the 15 degree threshold will build a motor learning pattern for preventive chronic neck injuries due to forward neck flexion. The University of Northern Illinois study by Ailneni, et. al (2017) evaluated the effects of wearing a posture sensor to conclude it had the ability to reduce physical demands on the neck. The study took 19 subjects who were otherwise healthy and had them wearing a posture sensor that would vibrate if the subject had his or her head in position at or greater than 15 degrees from neutral position for longer than 30 seconds. Subjects were asked to type continuously for 30 minutes with a 5 minute breaks with a control group and a study group. The subjects worked for a total of 8 hours to replicate an average work day. The study concluded that on average there was an 8% reduction of lower neck flexion and a 14% reduction of excess weight put on the neck from awareness of posture. Straker, et. Al. (2008) looked into the effects of children's posture with regards to using tablets. The study consisted of 18 subjects aged 5 to 6 years old who were otherwise healthy and split into groups where they would do coloring on a desktop computer, tablet or on paper. The paper and tablet showed to have a greater angle of neck flexion than that of the desktop computer.

While Google's glasses have attempted to take the hand-held electronic device out of the user's hand or lap and put the screen very close to the eye, it was noted that this approach creates additional concerns including potential brain radiation effects through the eye. The eye shields the brain the least from this radiation because there is no bone or other obstruction to impede brain access, In addition, the wearablity concerns has caused adoption rates to be infinitesimal. At the moment, the development on this type of device has virtually halted.

Consequently, to date, the primary concerns of most regarding mobile handheld devices has centered on the electromagnetic radiation effects of mobile phones when in contact close with the human body (head and mid torso). Combined with increasing usage of mobile phones across the human population has brought about active discussion and increased research on the impact of mobile phones on the social development and personal interaction skills of the hundreds of million active users, while significantly less attention is being given to the impact of mobile phones on the cervical posture of the human population at large, and health consequences this is likely to have.

Therefore, there is a great need in the art for a new and improved apparatus and method for promoting correct cervical spinal posture in both children and adults when using handheld electronic devices while overcoming the shortcomings and drawback of the prior art, which has primarily been focused on desktop computers.

### OBJECTS AND SUMMARY OF THE PRESENT INVENTION

Accordingly, it is a primary object of the present invention to provide a new and improved wearable apparatus and method for promoting correct cervical spinal posture in both children and adults when using electronic media, particularly handheld devices, while overcoming the shortcomings.

Another object of the present invention is to provide such apparatus in the form of a bio-engineered ultra-lightweight wearable device for children and adults alike to help reduce the effects that mobile handheld device use places on the human musculoskeletal structure, and more particularly, to reduce the impact of forward flexion of the human neck and head when using such handheld mobile devices.

Another object of the present invention is to provide a new and improved method of limiting forward neck flexion when viewing a handheld electronic device.

Another object of the present invention is to provide a new wearable bio-engineered device that has been designed and engineered to be worn beneath the chin of a human wearer for the purpose of limiting forward neck flexion when the wearer is viewing a handheld electronic devices and portable computers.

Another object of the present invention is to provide such a bio-engineered device comprising a support frame having a pair of spaced apart support arm members that gently engage about the sides of the human neck of the wearer, while supporting a chin support member disposed beneath the chin and above the chest of the wearer, wherein the support frame and the chin support member are configured and sized so that when the head of the human wearer flexes downward towards the chest of the wearer while viewing the handheld mobile device, the chin support member is positioned directly between the chin and chest of the wearer and prevents the head from flexing beyond a predetermined limit on flexion set by the length dimensions of the spaced apart support arm members and the height dimension of the chin support member.

Another object of the present invention is to provide such a wearable bio-engineered device that can be quickly and simply installed and comfortable to wear over extended periods of time when the forward flexion of the head is most apparent and necessary.

Another object of the present invention is to provide a method of protecting the spinal column of a human by wearing a device beneath the chin of the wearer, and above the chest of the wearer, which prevents the wearer from flexing ones neck and tiling ones head beyond a predetermined threshold flexion angle that is physically constrained by a chin support member that comfortably is disposed between the chin and the chest of the human wearer, and supported by a pair of comfortable support arms that gentle embrace the neck of the human wearer.

These and other objects of the present invention will become apparent hereinafter and in the Claims to Invention.

### BRIEF DESCRIPTION OF THE DRAWINGS

The following Objects of the Present Invention will become more fully understood when read in conjunction of the Detailed Description Of The Illustrative Embodiments, and the appended drawings, wherein:

FIGS. 1A, 1B, 1C, 1D and IE show a series of prior art cross-sectional side human skeletal cross-sectional views, illustrating various positions when the human head is tilted from 0 to 60 degrees of neck/head flexion angle, while viewing a handheld mobile device such as a smartphone, and generating strain on the spinal vertebrae along the neck of the human skeletal system;

FIG. 2A is a prior art cross-sectional side skeletal view of the upper portion of a human, showing the natural alignment of the chin when the human head is held in an upright posture;

FIG. 2B is a prior art cross-sectional side skeletal view of the upper portion of a human, showing the natural alignment of the chin, relative to the chest bone when the human head is tilted down towards the chest when viewing a hand-held mobile device, or a laptop device, as the case may be, creating stress and strain on the vertebrae along the neck and spinal column;

FIGS. 3A and 3B show prior art side views of a human viewing a laptop computer system on a desktop at different viewing angles, illustrating how the human head is tilted, during viewing and creating stress and strain along the vertebrae of the human neck and spinal column;

FIG. 4A is a first perspective view of the wearable bio-engineered device of the present invention designed to be worn beneath the chin of the human wearer for the purpose of limiting forward neck flexion when the wearer is viewing handheld electronic mobile devices or other portable computers or viewing another media display device, wherein the device comprises a support frame having a pair of spaced apart support arm members that gently engage about the sides of the human neck of the wearer, while supporting a chin support member disposed beneath the chin and above the chest of the wearer, and configured and sized so that when the head of the human wearer flexes downward towards the chest of the wearer while viewing the handheld mobile device, the chin support member is positioned between the chin and chest of the wearer and physically constrains and prevents the neck from flexing and head from tilting beyond a predetermined flexion limit set by the physical length dimensions of the spaced apart support arm members and the physical height dimension of the chin support member;

FIG. 4B is a plan view of the wearable bio-engineered device of the present invention shown in FIG. 4A;

FIG. 4C is an elevated side view showing the wearable device of the present invention shown in FIGS. 4A and 4B, showing the pair of spaced apart support arm members, provided with cushioning material;

FIG. 4D is a perspective view of the wearable bio-engineered device of the present invention showing the chin support member, mounted about the middle portion of the support frame, and also provided with cushioning material, to provide comfort and long-wearing characteristics;

FIG. 5 is a side cross-sectional skeletal view of a human who is being sized for a wearable bio-engineered device according to the present invention, showing the capture of three essential parameters to be measured from the wearer, for use in selecting the correct sized device from the sizing table shown in FIG. 7;

FIG. 6A is a front view of the wearable bio-engineered device of the present invention, showing device parameters (D and F) relating to the chin support member of the device;

FIG. 6B is a side view of the wearable bio-engineered device of the present invention, showing device parameters (E) relating to the chin support member of the device;

FIG. 7 is a sizing chart developed for use with the wearable bio-engineered device of the present invention, and showing what the general physical dimension requirements are likely to be for a given wearable device, given the three essential parameter measurements made on the particular human who is planning on wearing the device for protection purposes;

FIG. 8A is a side cross-sectional skeletal view of the human sized, fitted and wearing a wearable device of the present invention, beneath their chin, for purposes of the present invention, while the head is held in its standard upright position α=0;

FIG. 8B is a side cross-sectional skeletal view of the human sized, fitted and wearing a wearable bio-engineered device of the present invention, beneath their chin, for purposes of the present invention, while the head is tilted to its extreme position α=20, and prevented from tilting beyond that extreme threshold flexion angle, forcing the wearable to view the electronic media devices within a safe viewing limits/range;

FIG. 9 is a side cross-sectional skeletal view of the human sized, fitted and wearing a wearable bio-engineered device of the present invention, beneath their chin, for purposes of the present invention, while viewing a handheld electronic device and portable computers, while the head is tilted to its extreme position α=20, and prevented from tilting beyond that extreme threshold flexion angle, forcing the wearable to view the electronic media devices within a safe viewing limits/range;

FIG. 10A shows a side view of a human wearing the wearable bio-engineered device of the present invention while viewing a hand-held mobile phone device, with the head flexion angle being approximately α=0;

FIG. 10B shows a side view of a human wearing the wearable bio-engineered device of the present invention while viewing a hand-held mobile phone device, with the head flexion angle being approximately α=10;

FIG. 10C shows a side view of a human wearing the wearable bio-engineered device of the present invention while viewing a hand-held mobile phone device, with the head flexion angle being approximately α=20; and

FIG. 11 is a flow chart describing the primary steps taken when practicing the method of preventing cervical spinal injuries using the wearable bio-engineered device of the present invention during prolong viewing of electronic display media such as mobile phones, laptops, television, and the like.

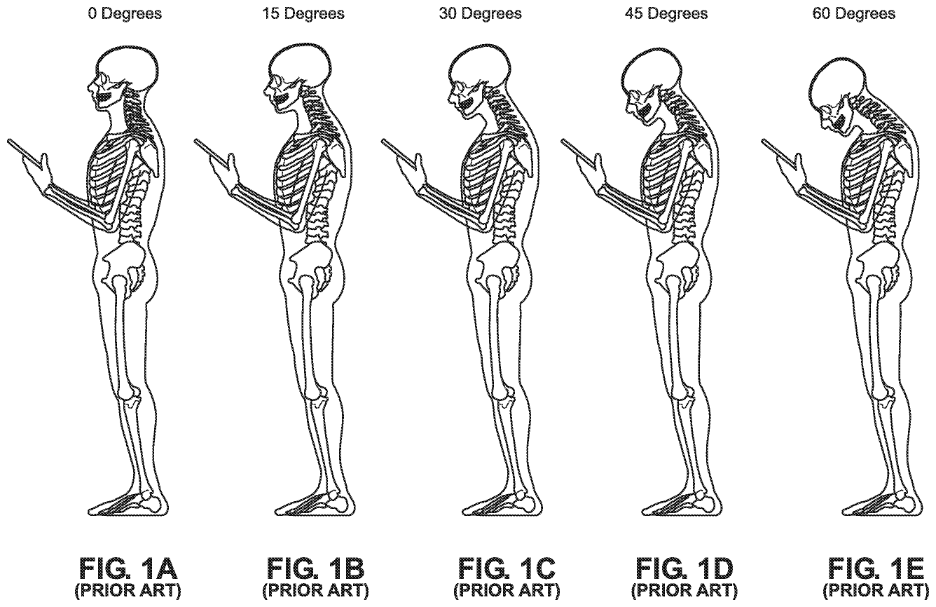

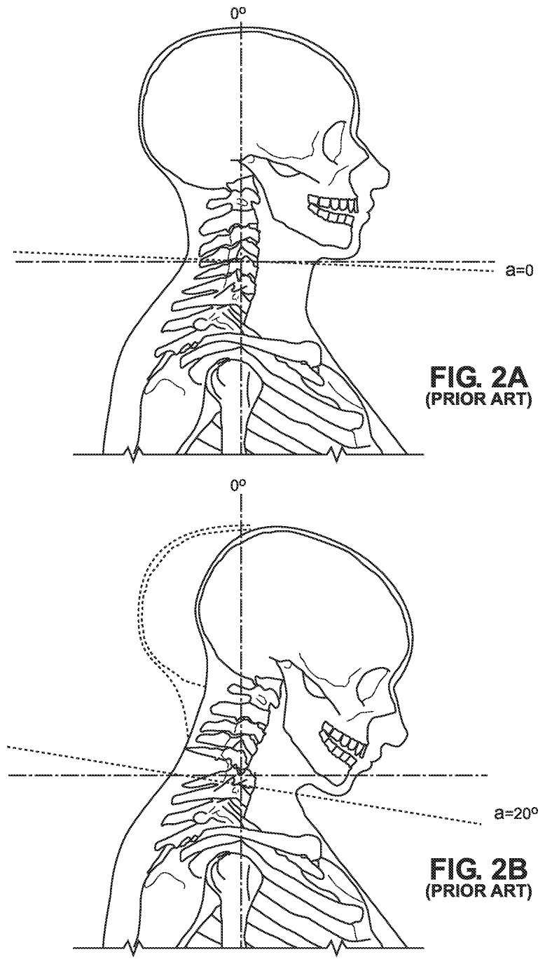

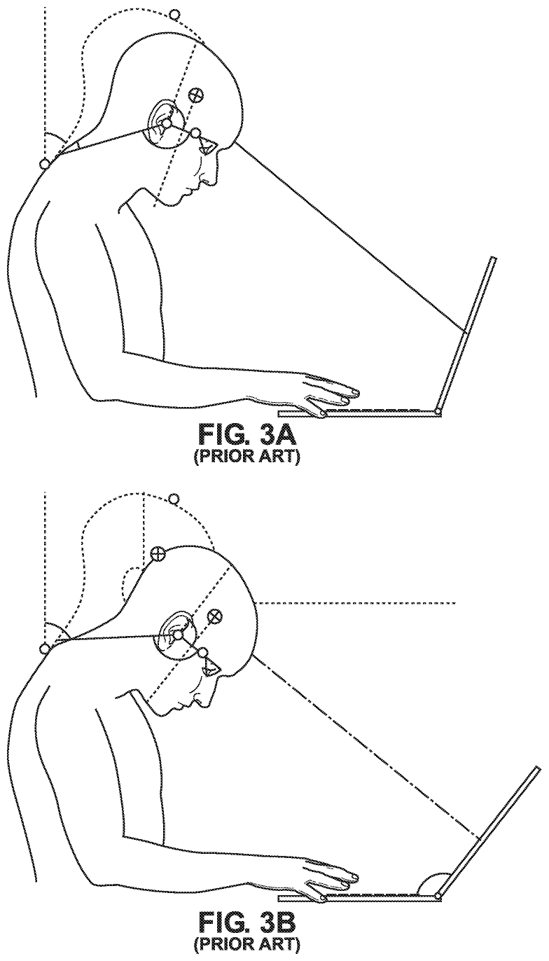

### DETAILED DESCRIPTION OF THE ILLUSTRATIVE EMBODIMENTS

### Overview of the Wearable Bio-Engineered Device and Method of the Present Invention

The present invention provides a novel method of and bio-engineered wearable device 1 that promises to provide preventive and therapeutic benefits to millions who are either currently or may in the future suffer from the increased use of handheld electronic devices 10, and adverse effects and physical demands such devices are having on human posture.

For adults and children, suffering from poor posture and stagnant positions with forward neck flexion often have chronic short and long term effects as a result of normal forward head flexion while using mobile devices 10 is typically greater than 12 degrees and up to 60 degrees.

Proper use of the present invention will promote, prevent and restrict excessive forward head flexion as well as creating a motor learning pattern. By virtue of the present invention, the general public stands to benefit in significant ways as will be described in greater detail herein.

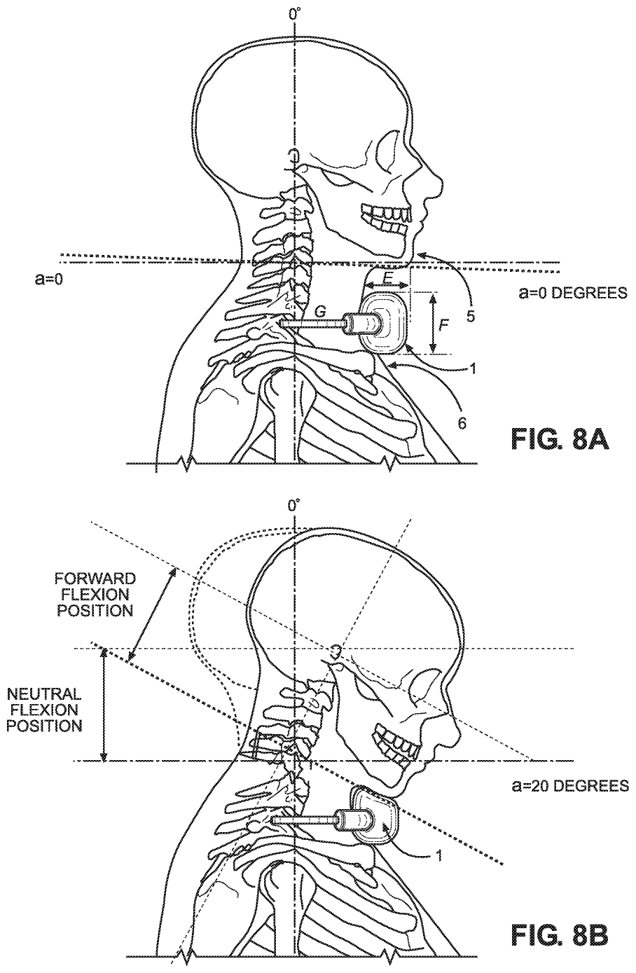

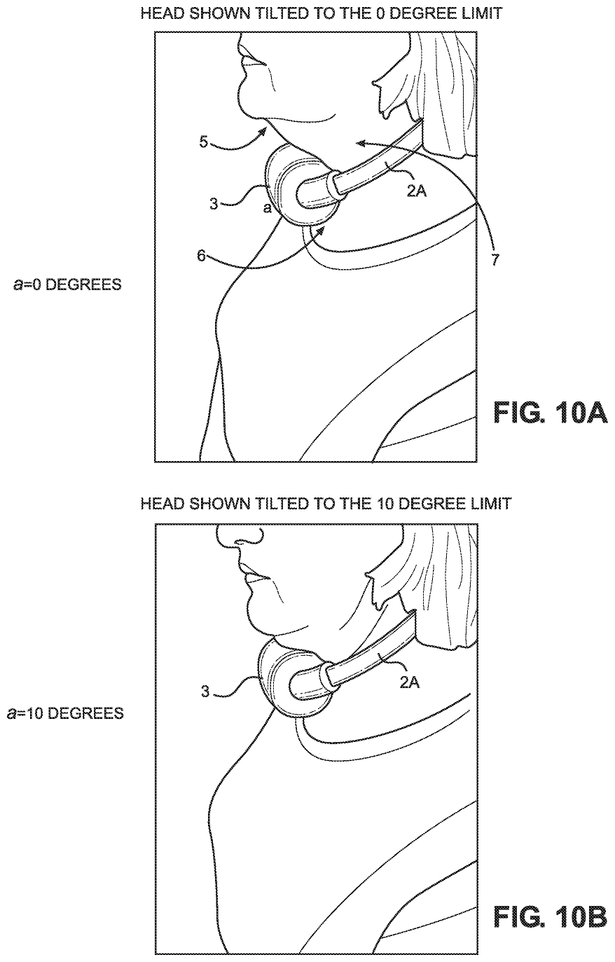

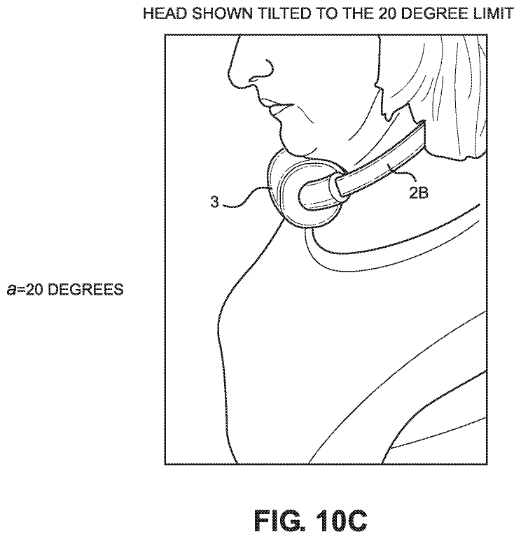

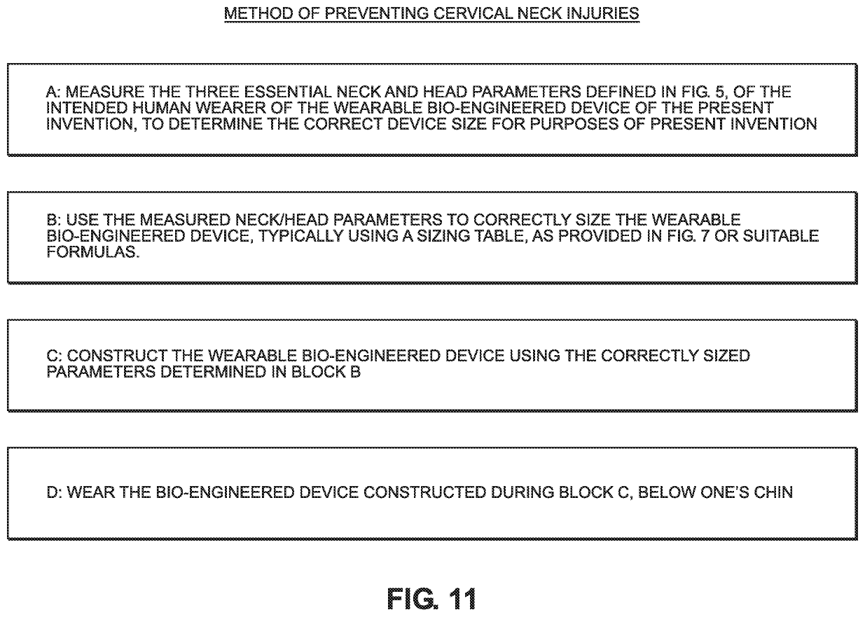

In general, and shown in FIGS. 8A through 8C, and 10A through 10C, the bio-engineered device of the present invention 1 is designed to be worn loosely around the human neck with a comfortable padding on its chin support member 3 so that, when then neck is flexed to accommodate the use of mobile devices at that +12 degree vulnerable angle, the person's chin 5 will gently touch the padding of the chin support member 3 at that approximate 12 degree forward flexion point. This stop point will serve to limit further forward neck flexion. Furthermore, this simple bio-feedback mechanism not only creates a motor learning pattern to promote better posture, but it also influences activating the correct muscles to ensure proper posture.

The present invention is radically different than any type of neck brace or neck support device, because neck braces are provided for the purpose of stability and reinforcement of a neck that has sustained a serious type of injury, requiring induced or controlled immobilization of the neck flexion, and the bio-engineered device of the present invention is not intended or bio-engineered for such problems and purposes. In contrast, bio-engineered device of the present invention 1 has been designed and engineered for the purpose of promoting and correcting posture by encouraging the activation of all postural muscles and at all times strengthening the same, to prevent future injury.

The bio-engineered device of the present invention 1 promotes healthier neck posture by helping the individual take 2 to 3 times the gravitational moment off the neck, thus leading to less current and future chronic neck issues. The bio-engineered device 1 is simple, affordable, and the ability to help millions fulfill a more productive, healthier lifestyle as the digital age continues to proliferate our daily lives.

Specification of the Wearable Bio-Engineered (i.e. Bio-Fitted) Device of the Present Invention

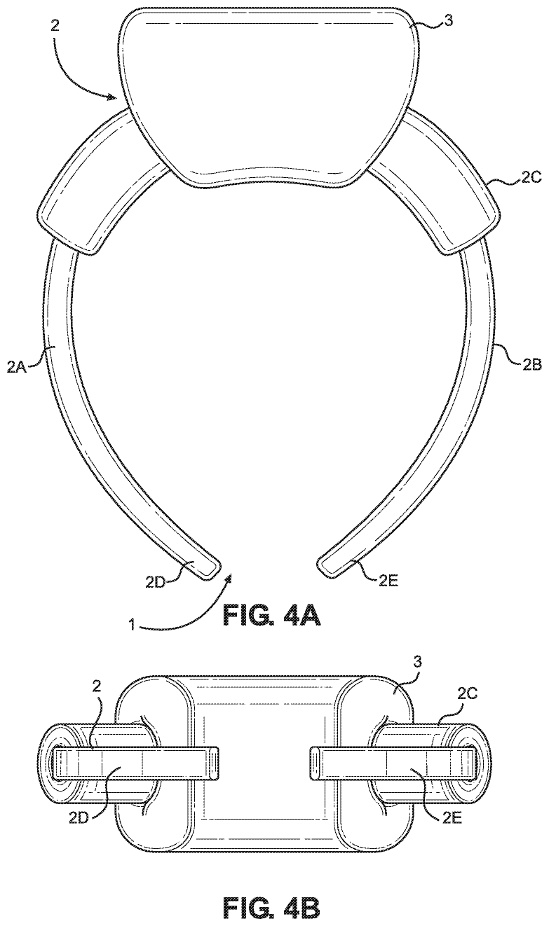

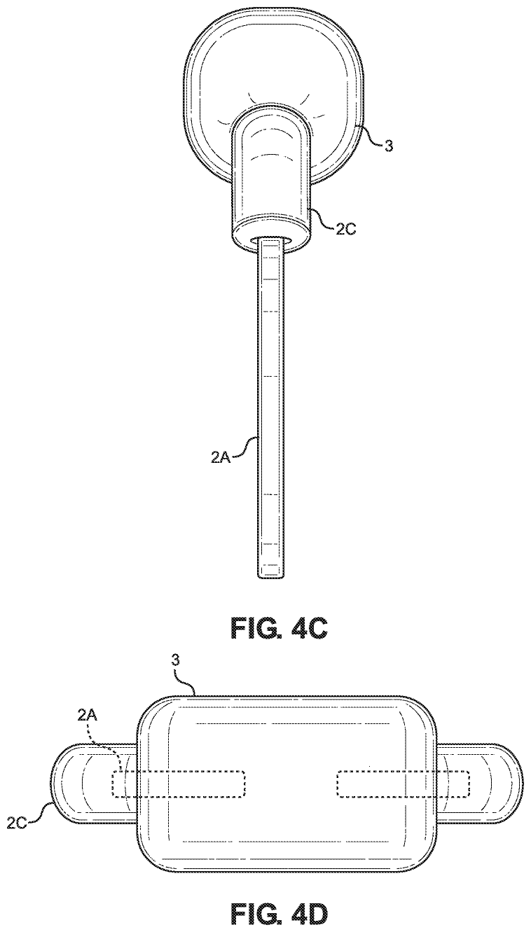

FIG. 4A shows the wearable bio-engineered device of the present invention 1 designed to be worn beneath the chin of the human wearer for the purpose of limiting forward neck flexion when the wearer is viewing a handheld mobile device or laptop on a desktop or viewing another media display device.

As shown in FIGS. 4A through 4D and 10A through 10C, the wearable device 1 comprises: a support frame 2 having a pair of spaced apart support arm members 2A and 2B that gently engage about the sides of the human neck 7 of the wearer, while supporting a chin support member 3 disposed beneath the chin 5 and above the chest 6 of the wearer, and configured and sized so that when the head of the human wearer flexes downward towards the chest of the wearer, while viewing the handheld mobile device 10, the chin support member 3 is positioned directly between the chin 5 and chest 6 of the wearer and physically constrains and prevents the neck from flexing and head from tilting beyond a predetermined flexion limit a set by the physical length dimensions of the spaced apart support arm members 2A, 2C and the physical height dimension (F) of the chin support member 3.

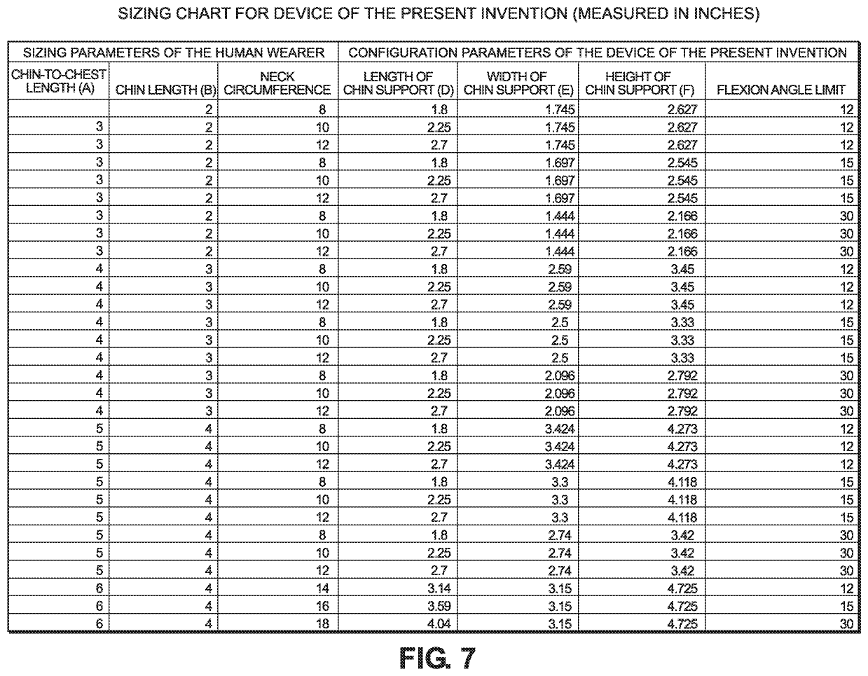

As shown in FIGS. 4A, 4B, 4C and 4D, the support frame 2 can be realized as plastic hoop-like structure, provided with a tube of cushioning material 2C (e.g. foam, fabric and/or plastic) supported about the mid-section of the support frame. Then about the support frame tube 2C, the chin support member 3, realizable from as a larger cushioning tube (e.g. foam, fabric and/or plastic), is mounted over the support tube 2C. The length of the chin support member (D), the width of the chin support member (E), and the height of the chin support member (F) will be determined based on the sizing parameters of the wearer using the sizing table of FIG. 7, and will vary in size from individual wearer to individual wearer, to meet the functional specifications of the bio-engineered device 1 as taught clearly herein, namely, to limit the neck/head flexion angle in a comfortable manner, without other restrictions, while using hand-held mobile phones, watching television, and watching other electronic media being displayed on all sorts of display screens appearing in our modern world.

As shown in FIGS. 4A through 4D and 10A through 10C, a soft and smooth fabric or foam covering material will be provided about the spaced-apart support arm members 2A and 2B of the support frame, to make wearing the bio-engineered (i.e. bio-fitted) device very comfortable, with application of forces to the neck surface that might make anyone feel uncomfortable. An advantage of the hoop-design of the support frame 2 of the present invention is that it allows for the use of minimal forces on about the wear's neck surface while supporting the chin support member in place between the wear's chin and chest surfaces, as shown and taught in the present invention.

In alternative designs, the support frame 2 might be provided with a hinged design, with or without biasing springs or elements to generate the gentle set of forces that will maintain the support frame 2 in place so that the chin support member 3 is maintained properly beneath the wear's chin and chest surface, as taught herein.

In alternative designs, it is also possible to realize the chin support member 3 as an inflatable bladder, inflated with air or other liquid materials to produce the physical dimensions required to provide the required support between the chin and chest surface that will maintain and restrict the neck/head flexion angle α to within its controlled threshold limit, determined empirically and set by design.

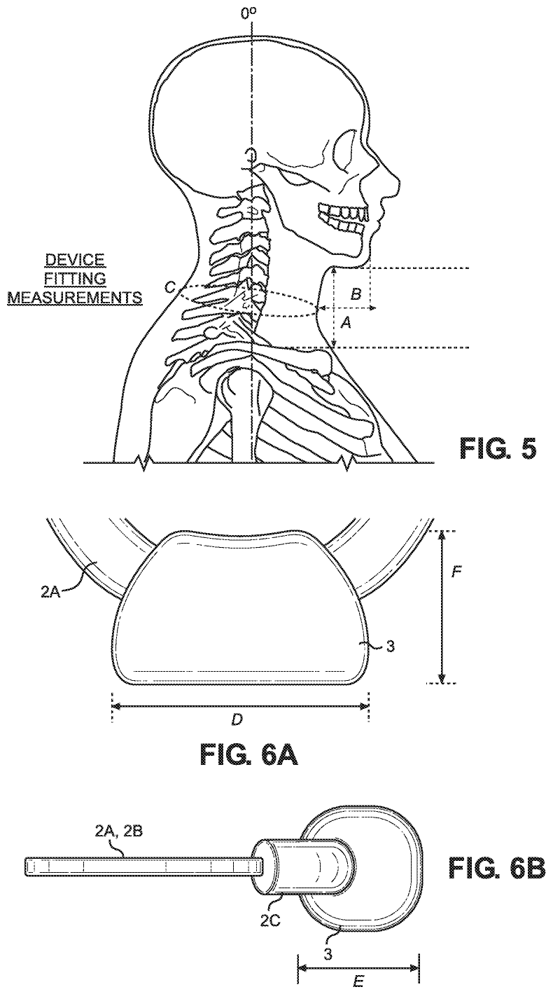

FIG. 5 illustrates a human who is being sized for a wearable bio-engineered (i.e. bio-fitted) device according to the present invention, showing the capture of three essential wearer parameters, namely, (i) The chin-to-chest length (A) parameter defined in FIG. 5, (ii) the chin length (B) parameter defined in FIG. 5; and (iii) the neck circumference (C) parameter defined in FIG. 5, to be measured from the wearer, for use in selecting the correct sized device from the sizing table shown in FIG. 7.

FIGS. 6A and 6B shown the wearable bio-engineered device of the present invention, with its device parameters (D, E and F) defined accordingly, namely: (i) the length of chin support (D) parameter specified in FIGS. 6A and 6B; (ii) the width of chin support (E) parameter specified in FIGS. 6A and 6B; the height of chin support (F) parameter specified in FIGS. 6A and 6B; and the neck/head flexion angle limit (e.g. α=20) specified in FIGS. 8A and 8B.

FIG. 7 shows a sizing chart developed for use with the wearable bio-engineered device of the present invention. This chart displays the general physical dimension/parameter requirements are likely to be for a given wearable device, is bio-correctly fitted/sized to the human wearer whose sizing parameters are specified by the three essential parameter measurements made on the particular human who is planning on wearing the device for protection purposes, namely: (i) a chin-to-chest length (A) parameter defined in FIG. 5 and measured as a the distance from the chin to the chest of the wearer, (ii) a chin length (B) parameter defined in FIG. 5 and measured as the length of the chin of the wearer, and (iii) a neck circumference (C) parameter defined in FIG. 5 and measured as the circumference of the neck of the wearer.

Using the three essential human wearer parameters (A, B, and C) specified above, which can be readily estimated from the wearer being fitted using a tape measure (or even finger estimations with conversion into length measurements (e.g. inches, centimeters, etc.), in some situations.

For a given set of these biologically-specified human wearer “sizing parameters”, the table of FIG. 7 or other mathematical methods based on geometry and/or algebra, can be used to determine the correct set of device “configuration parameters” for a device that his correctly bio-fitted to the body-sized of the individual wearer, namely: (i) the length of chin support (D) parameter specified in FIGS. 6A and 6B; (ii) the width of chin support (E) parameter specified in FIGS. 6A and 6B; the height of chin support (F) parameter specified in FIGS. 6A and 6B; and the neck/head flexion angle limit (e.g. α=20) specified in FIGS. 8A and 8B, to be maintained and controlled (not to be exceeded) then wearing and using the bio-engineered wearable device of the present invention 1.

FIG. 8A shows the wearable bio-engineered device of the present invention worn on a human subject, beneath their chin, for purposes of the present invention, while the head is held in its standard upright position α=0.

FIG. 8B shows the wearable bio-engineered device of the present invention, when worn on the human subject, beneath their chin, while the head is tilted to its extreme position α=20, and prevented from tilting beyond that extreme threshold flexion angle, forcing the user to view the electronic media devices within a safe viewing limits/range.

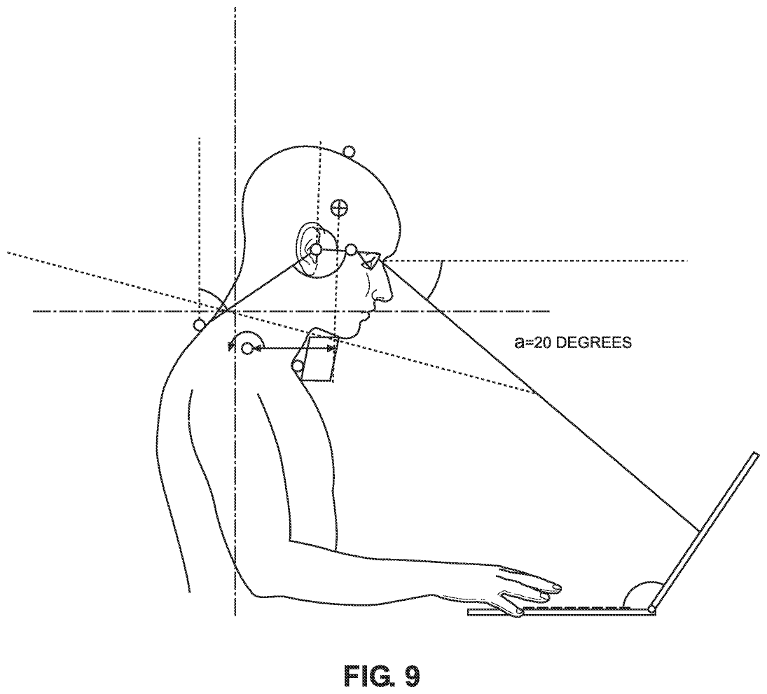

FIG. 9 illustrates how the wearable bio-engineered device of the present invention, when worn beneath the human subject's chin, for purposes of the present invention, prevents the head from tilting beyond that extreme threshold flexion angle, forcing the user to view the electronic media devices within a safe viewing limits/range.

Referring to FIG. 11, the method of preventing cervical spinal injuries during prolonged viewing of electronic media display devices, will now be described in detail below.

As indicated at Block A in FIG. 11, the first step of the method according to the principles of the present invention involves measuring or estimating the three essential wearer parameters, involving the neck and head of the intended wearer of the device, as defined in FIG. 5. These parameters are: (i) The chin-to-chest length (A) parameter defined in FIG. 5, (ii) the chin length (B) parameter defined in FIG. 5; and (iii) the neck circumference (C) parameter defined in FIG. 5, to be measured from the wearer.

As indicated at Block B in FIG. 11, the method involves using the wearer's measured neck/head parameters to estimate a corresponding set of “configuration parameters” for the properly sized the bio-engineered device 1 to function as intended in accordance with the present invention. As taught above, these configuration parameters are: (i) the length of chin support (D) parameter specified in FIGS. 6A and 6B; (ii) the width of chin support (E) parameter specified in FIGS. 6A and 6B; the height of chin support (F) parameter specified in FIGS. 6A and 6B; and the neck/head flexion angle limit (e.g. α=20) specified in FIGS. 8A and 8B, to be maintained and controlled (not to be exceeded) then wearing and using the bio-engineered wearable device 1.

During Block B in FIG. 11, the method may involves using a table containing proper design parameters as shown in FIG. 7, or mathematical formulas and a computing device, to determine the correctly sized components of the device for any particular human wearer (e.g. child and adult alike), and from these correct components, a bio-correctly size device can be constructed for wearing by the human subject, that matches his or her skeletal structure at the timing of wearing. The computing device may be a virtual kiosk provided online or a physical kiosk provided in a retail store assisting consumes and sales clerks in the correct sizing of the wearable bio-engineered device of the present invention 1.

As indicated at Block C in FIG. 11, the determined/estimate configuration parameters are then used to construct or otherwise procure a real operable wearable device fit for the intended user.

While a table based method of device design and construction is illustrated in FIG. 7, it is understood that it is possible to adapt this method into a size-based method there quantized sizes are predesigned and preconstructed for different sized human wearers, and maintained in inventory, much like shoe sizing methods currently in practice around the world. In the table of FIG. 7, the sizes shown can be grouped by small parameter ranges into different sizes. This would simplify bio-correct fitting of the device of the present invention within the spirit of the present invention, while reducing costs of manufacture and delivery to consumers.

As indicated at Block D in FIG. 11, after construction or procurement, the bio-correct device is then worn on the human about her/his neck, and under the chin as shown in FIGS. 10A through 10C.

FIG. 10A shows the human wearing the wearable bio-engineered device of the present invention 1 while viewing a hand-held mobile phone device, with the head flexion angle being approximately α=0.

FIG. 10B shows the human wearing the wearable bio-engineered device of the present invention 1 while viewing a hand-held mobile phone device, with the head flexion angle being approximately α=10.

FIG. 10C shows the human wearing the wearable bio-engineered device of the present invention 1 while viewing a hand-held mobile phone device, with the head flexion angle being approximately α=20, the threshold angle in most user applications.

As shown in FIGS. 10A through 10C, the wearable bio-engineered device of the present invention, when worn about the neck of a human subject, safely restricts forward neck flexion to an appropriate ˜12 degree angle before comfortably and safely inhibiting further movement beyond this threshold limit. The device of the present invention limits forward neck flexion, and also assists as a training aid to promote correct posture position when using a mobile device so that an individual will develop proper muscle memory assistance in using handheld devices thus limiting the forward neck flexion

By virtue of the present invention, it is possible to reduce cervical spine injuries and musculoskeletal disorders that may are likely to develop through long term ongoing use of hand-held mobile devices and portable computers.

### Modifications to the Present Invention which Readily Come to Mind

The illustrative embodiment discloses the use of a novel device for preventing cervical spine injuries potentially occurring through prolonged use of handheld mobile devices.

In the illustrative embodiment, plastic support arms were used to support the chin support member which is preferably made from a hard foam material. It is understood that other materials and fabrication methods can be used to fabricate the device.

These and other variations and modifications will come to mind in view of the present invention disclosure.

While several modifications to the illustrative embodiments have been described above, it is understood that various other modifications to the illustrative embodiment of the present invention will readily occur to persons with ordinary skill in the art. All such modifications and variations are deemed to be within the scope and spirit of the present invention as defined by the accompanying Claims to Invention.

What is claimed is:

1. A wearable device for preventing cervical spine injuries in humans, comprising:
a support frame having a pair of spaced apart support arm members that gently engage about the sides of the human neck of a human wearer; and
a chin support member supported by said support frame and disposed beneath the chin and above the chest of the human wearer,
wherein said support frame and said chin support member are configured and sized so that when the head of the human wearer flexes downward towards the chest of the wearer, while viewing the handheld mobile device, the chin support member is positioned between the chin and chest of the wearer and physically constrains and limits said neck from flexing and head from tilting beyond a predetermined flexion limit set by the physical length dimensions of the spaced apart support arm members and the physical height dimension of said chin support member.

2. The wearable device of claim 1, wherein said wearable device is bio-fitted to the individual user using wearer parameters selected from the group consisting of (i) a chin-to-chest length (A) parameter measured as a the distance from the chin to the chest of the wearer, (ii) a chin length (B) parameter measured as the length of the chin of the wearer, and (iii) a neck circumference (C) parameter measured as the circumference of the neck of the wearer, for use in selecting the correct sized device.

3. The wearable device of claim 2, wherein said wearable device is bio-fitted/sized to the individual wearer using said wearer parameters that are matched to a set of sizing parameters of said wearable device selected from the group consisting of (i) the length of said chin support member, (ii) the width of said chin support member, and (iii) the height of said chin support member.
